(19) United States
(12) Patent Application Publication
(10) Pub. No.: US 2025/0371083 A1
(43) Pub. Date: Dec. 4, 2025

(54) SYSTEM AND METHOD FOR LLM-BASED USER INTERFACES FOR ANALYSIS OF MEDICAL MATERIALS

(71) Applicant: KLICK INC., Toronto (CA)

(72) Inventors: Kamran SHAH, Toronto (CA); Nu Ngoc DUONG, Toronto (CA)

(73) Assignee: KLICK INC., Toronto (CA)

(21) Appl. No.: 19/208,069

(22) Filed: May 14, 2025

Related U.S. Application Data
(60) Provisional application No. 63/654,561, filed on May 31, 2024.

(51) Int. Cl. G06F 16/951; G06Q 30/0242; G16H 50/70

(52) U.S. Cl. CPC G06F 16/951; G06Q 30/0243; G16H 50/70

(57) ABSTRACT

Provided are computer-implemented methods and systems for generating an large language model (LLM)-based user interface for a user at a user device, including: providing, a memory comprising a database, the database comprising at least one historical content version and at least one criteria prompt; automatically transmitting, at a network device, a content collection request; receiving, at the network device based on the content collection request, a content collection response comprising an updated content version; generating, at a processor in communication with the memory and the network device, an LLM request comprising the at least one historical content version, the updated content version, and the at least one criteria prompt; transmitting, from the network device to an LLM system, the LLM request; receiving, at the network device from the LLM system, an LLM response; and generating, at the processor, a user interface for content analysis based on the LLM response.

### CROSS-REFERENCE TO RELATED APPLICATIONS

The present application claims priority to U.S. provisional application 63/654,561 filed May 31, 2024 the entire contents of which are incorporated herein by reference.

### FIELD

The present embodiments are directed generally to large-language model (LLM) based user interfaces for analyzing medical materials, and specifically, LLM-based user interfaces for automatically evaluating a pharmaceutical product or medical device disclosure such as through a website.

### BACKGROUND

Pharmaceutical companies are required to follow complex regulatory rules governing how they promote their products. For example, the United States Food and Drug Administration (FDA) requires that any statement made about the benefits of a drug be supported by two independently-conducted double-blind placebo-controlled trials of adequate statistical power or equivalent evidence, and include fairly balanced information about risks. Thus, this content is required to comply with applicable regulatory, statutory, or ethical requirements.

The publishing of medical materials associated with a pharmaceutical or a medical device, i.e., printer materials, websites, advertisements, marketing materials, therefore require close tracking and approval of changes that are published. First, from the perspective of compliance concerns with applicable regulatory, statutory, or ethical requirements, and second, as a matter of performance analysis of the content in view of competitors.

Conventional marketing materials relied on printed materials. With printed materials, long review cycles and the lead times associated with printing and distribution mean that the review of these materials is typically done in batches. The review of these materials was performed on a long cycle and many changes would be made in a single cycle.

In the modern digital environment, changes to these materials may be published frequently—in some cases multiple times per day in the case of product information websites. This frequency of changes cause problems that differ from conventional marketing including the use of printed materials. These problems include a lack of automated solutions, a lack of fast feedback of the digital content in order to facilitate the fast publishing cycle, and a lack of an ability to compare marketing messages of competitors operating on a similarly short publishing scale. The time scales encountered are further exacerbated by the delivery of content through social media posts, websites, videos, and other digital materials delivered using a computer.

Separately, many firms and their competitors may deliver content using an A/B testing framework that may mean that some recipients of the marketing materials may see different content from others if they are randomly assigned into the test group.

Improvements are therefore desired to address these computer-based problems in order to provide user interfaces for analysis of medical materials.

### SUMMARY

Provided are systems and methods for automatically capturing digital content associated with a pharmaceutical product or medical device, assessing it in view of competitor products, and providing a user interface related to the digital content intended to automatically provide analysis and feedback to an editor to improve their ability to get timely information related to their marketing of the product or device.

In this manner, authors of promotional materials have reduced cost for the publication of digital materials, and get more timely information about changes in the digital content automatically, both in view of changes by the manufacturer in their digital content and also in view of changes from competitors in their digital content.

In a first aspect, there is provided a computer-implemented method for generating an large language model (LLM)-based user interface for a user at a user device, comprising: providing, a memory comprising a database, the database comprising at least one historical content version and at least one criteria prompt; automatically transmitting, at a network device, a content collection request; receiving, at the network device based on the content collection request, a content collection response comprising an updated content version; generating, at a processor in communication with the memory and the network device, an LLM request comprising the at least one historical content version, the updated content version, and the at least one criteria prompt; transmitting, from the network device to an LLM system, the LLM request; receiving, at the network device from the LLM system, an LLM response; and generating, at the processor, a user interface for content analysis based on the LLM response.

In one or more embodiments, the method may further comprise: transmitting, using the network device, the user interface to the user device.

In one or more embodiments, the transmitting of the content collection request and the receiving of the content collection response may be performed using a web scraper.

In one or more embodiments, the web scraper may receive text-based data and image-based data in the content collection response.

In one or more embodiments, the method may further comprise generating, using the web-scraper, a screenshot.

In one or more embodiments, the method may further comprise storing, in the database, the screenshot, the text-based data, and the image-based data as a new historical content version, the new historical content version indexed with a timestamp.

In one or more embodiments, a first content version of the at least one historical content version may be associated with a first timestamp, and the updated content version is associated with a second timestamp.

In one or more embodiments, the method may further comprise: comparing, at the processor, the first content version and the updated content version to determine a content difference; and wherein the generating at the processor, the LLM request may comprise the content difference.

In one or more embodiments, the user interface may comprise a scorecard user interface, the scorecard user interface comprising at least one scorecard metric for each of the at least one criteria prompt, the scorecard user interface updated automatically based on the received updated content version.

In one or more embodiments, the user interface may comprise a checklist user interface, the checklist user interface comprising at least one checklist metric for each of the at least one criteria prompt, the checklist user interface updated automatically based on the received updated content version.

In a second aspect there is provided a system for generating an large language model (LLM)-based user interface for a user at a user device, comprising: a memory comprising a database, the database comprising at least one historical content version and at least one criteria prompt; a network device for: transmitting a content collection request; receiving a content collection response based on the content collection request, the content collection response comprising an updated content version; transmitting to an LLM system, an LLM request; and receiving from the LLM system, an LLM response, and a processor in communication with the memory and the network device, the processor configured to: generate the LLM request comprising the at least one historical content version, the updated content version, and the at least one criteria prompt; generate a user interface for content analysis based on the LLM response.

In one or more embodiments, the processor may be further configured to: transmit, using the network device, the user interface to the user device.

In one or more embodiments, the transmitting of the content collection request and the receiving of the content collection response may be performed using a web scraper.

In one or more embodiments, the web scraper may receive text-based data and image-based data in the content collection response.

In one or more embodiments, the processor may be further configured to generate, using the web-scraper, a screenshot.

In one or more embodiments, the processor may be further configured to store, in the database, the screenshot, the text-based data, and the image-based data as a new historical content version, the new historical content version indexed with a timestamp.

In one or more embodiments, a first content version of the at least one historical content version may be associated with a first timestamp, and the updated content version is associated with a second timestamp.

In one or more embodiments, the processor may be further configured to: compare the first content version and the updated content version to determine a content difference; and wherein the processor is further configured to generate the LLM request comprises the content difference.

In one or more embodiments, the user interface may comprise a scorecard user interface, the scorecard user interface comprising at least one scorecard metric for each of the at least one criteria prompt, the scorecard user interface updated automatically based on the received updated content version.

In one or more embodiments, the user interface may comprise a checklist user interface, the checklist user interface comprising at least one checklist metric for each of the at least one criteria prompt, the checklist user interface updated automatically based on the received updated content version.

### DRAWINGS

FIG. 1 shows a system diagram for generating a large language model (LLM)-based user interface in accordance with one or more embodiments.

FIG. 2 shows a device diagram for the server 106 in FIG. 1 in accordance with one or more embodiments.

FIGS. 3A-3E show a user interface diagram in accordance with one or more embodiments.

FIGS. 4A-4F show another user interface diagram in accordance with one or more embodiments.

FIGS. 5A-5E show another user interface diagram in accordance with one or more embodiments.

FIG. 6 shows another user interface diagram in accordance with one or more embodiments.

FIG. 7 shows a method diagram in accordance with one or more embodiments.

FIGS. 8 and 9 show content profile configuration examples in accordance with one or more embodiments.

### DESCRIPTION OF VARIOUS EMBODIMENTS

Various embodiments will now be described below to provide an example of the claimed subject matter. No example described below limits any claimed subject matter and any claimed subject matter may cover embodiments such as systems or methods that differ from those described below.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures, and components have not been described in detail so as not to obscure the examples described herein. Also, the description is not to be considered as limiting the scope of the examples described herein.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Some elements herein may be identified by a part number, which is composed of a base number followed by an alphabetical or subscript-numerical suffix (e.g., 112a, or 112₁). Multiple elements herein may be identified by part numbers that share a base number in common and that differ by their suffixes (e.g., 112₁, 112₂, and 112₃). All elements with a common base number may be referred to collectively or generically using the base number without a suffix (e.g., 112).

The example systems and methods described herein may be implemented in hardware or software, or a combination of both. In some cases, the examples described herein may be implemented, at least in part, by using one or more computer programs, executing on one or more programmable devices comprising at least one processing element, a data storage element (including volatile and non-volatile memory and/or storage elements), and at least one communication interface. These devices may also have at least one input device (e.g., a keyboard, a mouse, a touchscreen, and the like), and at least one output device (e.g., a display screen, a printer, a wireless radio, and the like) depending on the nature of the device. For example, and without limitation, the programmable devices (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some examples, the communication interface may be a network communication interface. In examples in which elements are combined, the communication interface may be a software communication interface, such as those for inter-process communication (IPC). In still other examples, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high-level procedural, declarative, functional or object-oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g., ROM, magnetic disk, optical disc) readable by a general or special purpose programmable computer, for configuring and operating the computer when the storage media or device is read by the computer to perform the procedures described herein. Examples of the system may also be considered to be implemented as a non-transitory computer-readable storage medium, configured with a computer program, where the storage medium so configured causes a computer to operate in a specific and predefined manner to perform the functions described herein.

Furthermore, the example system, processes and methods are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various examples of systems, methods and computer programs products are described herein. Modifications and variations may be made to these examples without departing from the scope of the invention, which is limited only by the appended claims. Also, in the various user interfaces illustrated in the figures, it will be understood that the illustrated user interface text and controls are provided as examples only and are not meant to be limiting. Other suitable user interface elements may be used with alternative implementations of the systems and methods described herein.

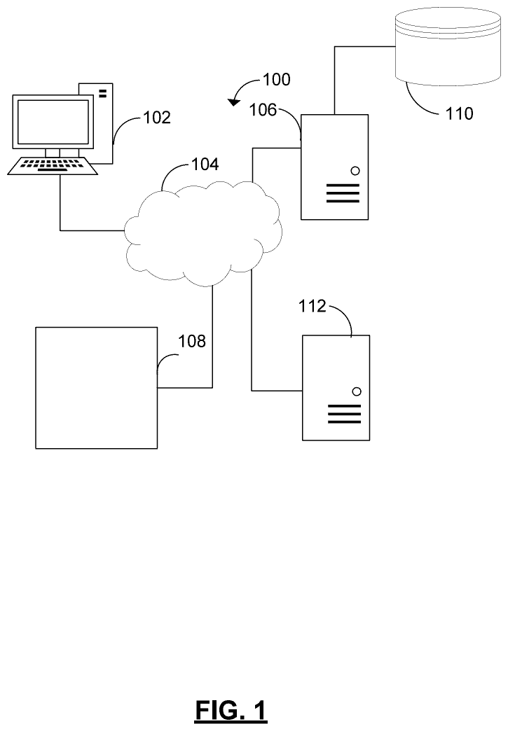

Reference is first made to FIG. 1 which shows a system 100 for generating a large language model (LLM)-based user interface in accordance with one or more embodiments.

The large language model (LLM)-based user interface generation system includes one or more user devices 102, a network 104, one or more servers 106 in communication with one or more databases 110, one or more LLM systems 108, and one or more content devices 112.

The one or more user devices 102 may be used by a user such as a consultant, a marketing professional, a medical professional, a creative professional, or any other intended end-user of a software application for generating or evaluating content for a pharmaceutical product or medical device. The user devices 102 are used to access an software application (not shown) running on server 106 over network 104 which may be provided by a downloadable application on user device 102 that connects to server 106, or using a web-based application in a browser on user device 102. The one or more user devices 102 may be any two-way communication device with capabilities to communicate with other devices. A user device 102 may be, for example, a mobile device such as mobile devices running the Google® Android® operating system or Apple® iOS® operating system. A user device 102 may also be, for example, a personal computer operating the Windows® or MacOS® operating system.

A user device 102 may be the personal device of a user or may be a device provided by an employer. The one or more user devices 102 may be used by a user to access the software application (not shown) running on server 106 over network 104. The user device 102 may be a desktop computer, mobile device, or laptop computer. The user device 102 may be in communication with server 106 and may allow a user to use the software application to perform actions such as creating or evaluating content related to pharmaceutical products or medical devices using LLM systems 108.

The application provided to the user either via an application at user device 102 or via a web interface from server 106 may include a user interface that accepts inputs in various forms, such as text or image.

The software application running on the one or more user devices 102 may communicate with server 106 using an Application Programming Interface (API) endpoint, and may send various inputs and other requests for processing at the one or more LLM systems 108.

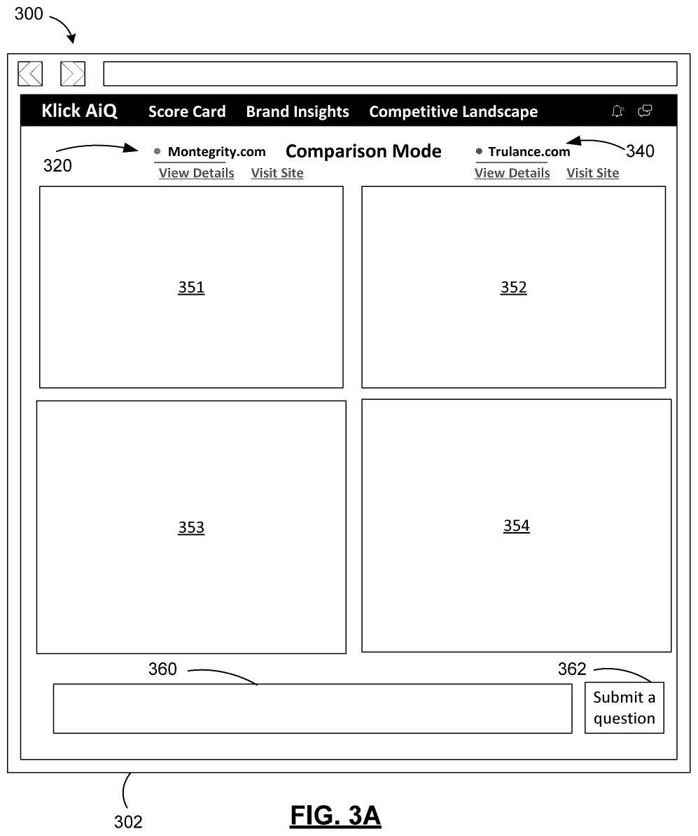

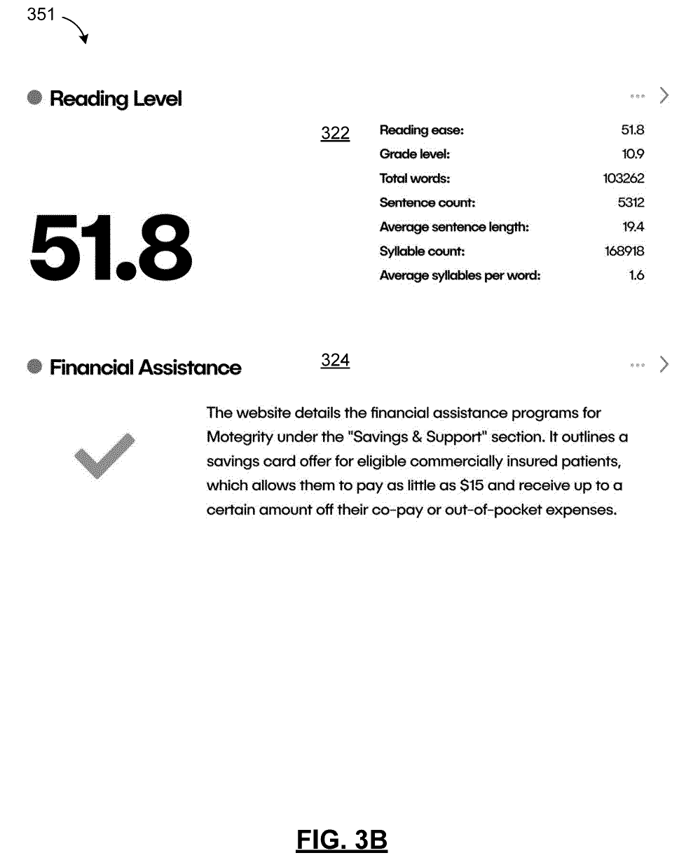

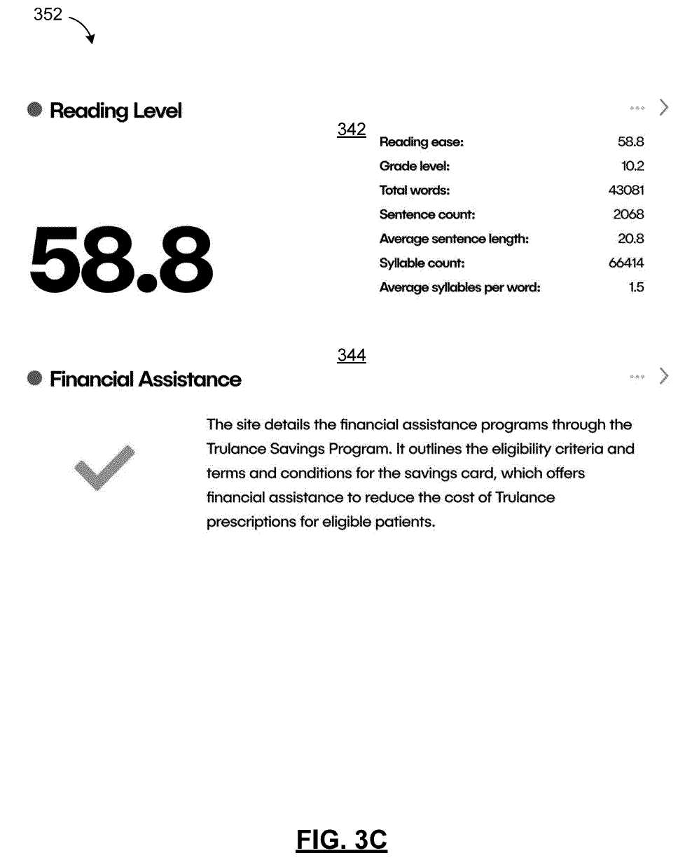

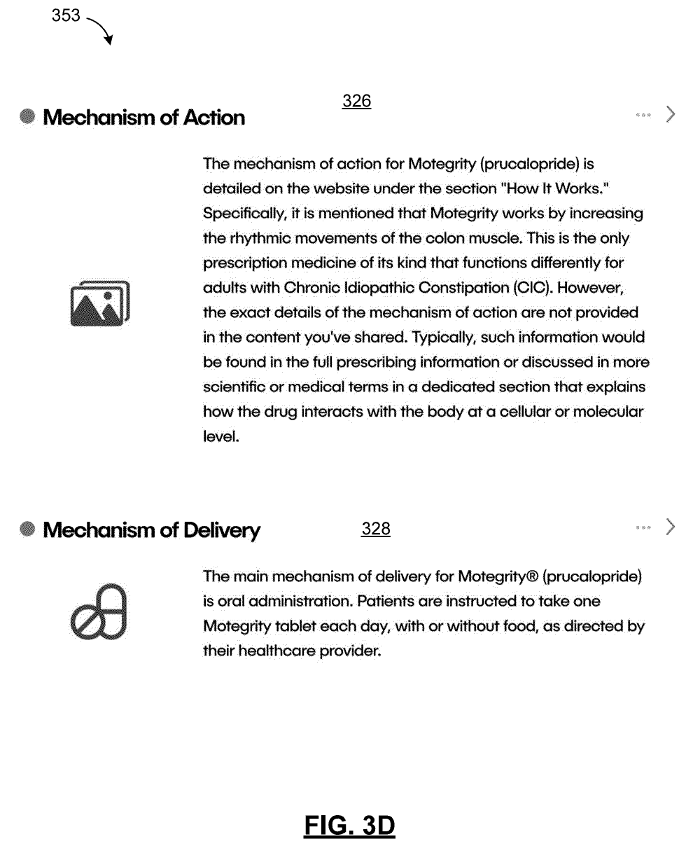

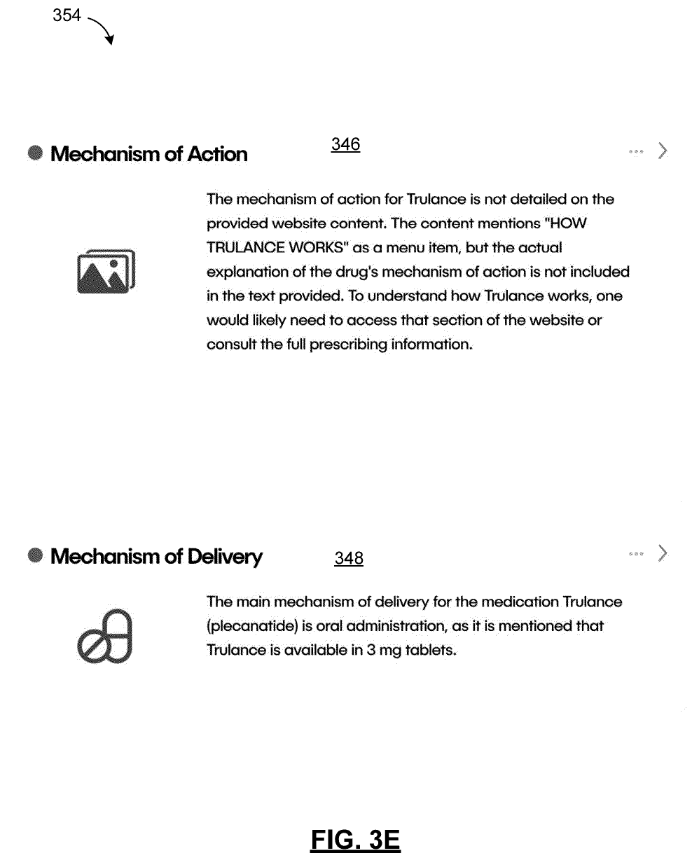

The software application running on the one or more user devices 102 may display one or more user interfaces on a display device of the user device, including, but not limited to, the user interface shown in FIGS. 3A-3E, 4A-4F and 5A-5E. A browser may be used at the user device 102 to access the web application running on server 106.

Network 104 may be any network or network components capable of carrying data including the Internet, Ethernet, fiber optics, satellite, mobile, wireless (e.g. Wi-Fi, WiMAX), SS7 signaling network, fixed line, local area network (LAN), wide area network (WAN), a direct point-to-point connection, mobile data networks (e.g., Universal Mobile Telecommunications System (UMTS), 3GPP Long-Term Evolution Advanced (LTE Advanced), Worldwide Interoperability for Microwave Access (WiMAX), etc.) and others, including any combination of these.

The server 106 is in network communication with the one or more user devices 102, the database 110, the one or more content devices 112, and the LLM system 108. The server 106 may host a web application or an Application Programming Interface (API) endpoint that the one or more user devices 102 may interact with via network 104. The requests made to the API endpoint of server 106 may be made in a variety of different formats, such as JavaScript Object Notation (JSON), extensible Markup Language (XML), or another known markup.

One or more LLM systems 108 are in network communication with the server 106. The LLM systems may be accessed by the server 106 using an API of the LLM systems 108. The LLM system 108 may use a pre-trained model (e.g. GPT-4 or similar) to process inputs from the user device 102 via the server 106 and produce output user interfaces for display on the user device 102. Detailed prompts and predefined crafted prompts provided by the server 106 may enable the LLM system 108 to adapt to the volume and type of input data without making any modifications to its base model. The one or more LLM systems 108 may include one or more Large Language Models (LLMs), one or more Image Generation Models (IGMs), and one or more Speech Models (SMs).

The one or more generative AI systems 108 may include one or more of the following:
GPT-2 made by OpenAI®
GPT-3 made by OpenAIR
GPT-3.5 Turbo made by OpenAI®
GPT-4 made by OpenAI®
GPT-4 Turbo made by OpenAIR
Claude 1 made by Anthropic®
Claude 2 made by Anthropic®
Claude 3 made by Anthropic®
BLIP made by Salesforce®
LaMDA (Language Models for Dialog Applications) made by Google®
LLaMA (Large Language Model Meta AI) made by Meta®
PaLM 2 (Pathways Language Model 2) Google®
Llama 2 made by Meta®
SDXL made by Stability AI®
Dall-E made by OpenAI®
Google Cloud Platform by Google®
ElevenLabs®
D-ID®

The database 110 may store historical content data, and configuration information for each of a plurality of products or devices. The database may be a Structured Query Language (SQL) such as PostgreSQL or MySQL or a not only SQL (NoSQL) database such as MongoDB. The database 110 may be a data lake that collects data published at the one or more content systems 112 and may further index the historical content.

The one or more content systems 112 may be public websites associated with a 1^{st} party pharmaceutical products or medical devices. The websites may be available on the public internet, or as part of a private network. The content systems 112 may include web servers and may be reachable using HTTP requests, or other network requests as known. The one or more content systems 112 may be for 3^{rd} party pharmaceutical products or medical devices. The content systems 112 may include public websites, websites for news publications, websites for product monographs, social media posts, YouTube® or other videos, clinical trials or clinical publications, etc.

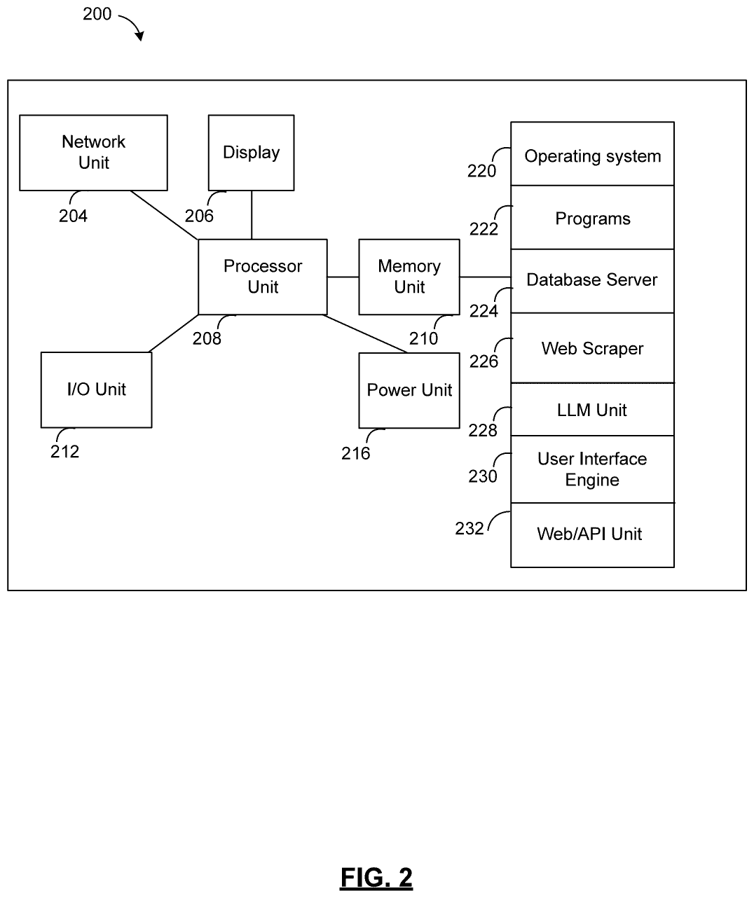

Referring next to FIG. 2, there is shown a server device diagram 200 for the server 106 in FIG. 1 in accordance with one or more embodiments.

The network unit 204 can include wired or wireless connection capabilities. The network unit 204 can include a radio that communicates using standards such as IEEE 802.11a, 802.11b, 802.11g, or 802.11n. The network unit 204 can be used by the server 200 to communicate with other devices or computers.

Network unit 204 may communicate with a network, such as network 104 (see FIG. 1).

The display 206 may be an LED or LCD based display and may be a touch sensitive user input device that supports gestures.

The processor unit 208 controls the operation of the server 200. The processor unit 208 can be any suitable processor, controller or digital signal processor that can provide sufficient processing power depending on the configuration, purposes and requirements of the server 200 as is known by those skilled in the art. For example, the processor unit 208 may be a high-performance general processor. In alternative embodiments, the processor unit 208 can include more than one processor with each processor being configured to perform different dedicated tasks. The processor unit 208 may include a standard processor, such as an Intel® processor or an AMD® processor.

The memory unit 210 comprises software code for implementing an operating system 220, programs 222, database server 224, web scraper 226, LLM unit 228, user interface engine 230 and web/API unit 232.

The memory unit 210 can include RAM, ROM, one or more hard drives, one or more flash drives or some other suitable data storage elements such as disk drives, etc. The memory unit 210 is used to store an operating system 220 and programs 222 as is commonly known by those skilled in the art.

The I/O unit 212 can include at least one of a mouse, a keyboard, a touch screen, a thumbwheel, a trackpad, a trackball, a card-reader, an audio source, a microphone, voice recognition software and the like again depending on the particular implementation of the server 200. In some cases, some of these components can be integrated with one another.

The power unit 216 can be any suitable power source that provides power to the server 200 such as a power adaptor or a rechargeable battery pack depending on the implementation of the server 200 as is known by those skilled in the art.

The operating system 220 may provide various basic operational processes for the server 200. For example, the operating system 220 may be a server operating system such as Ubuntu® Linux, Microsoft® Windows Server® operating system, or another operating system.

The programs 222 include various user programs. They may include several hosted applications delivering services to users over the network, for example, a marketing customer relations management (CRM) system.

In one or more embodiments, the programs 222 may provide a marketing or advertising platform that is web-based, or client-server-based application via Web/API Unit 232 that provides for the creation and evaluation of marketing content.

The database server 224 may be the historical content database 110 (see e.g., FIG. 1) for storing data associated with marketing content. The historical content in database 224 may be collected by web scraper 226.

The database server 224 may provide a data lake including content from content systems 112 (see e.g. FIG. 1) which may include a plurality of content profiles, each content profile associated with a particular pharmaceutical product or medical device. Each content profile may include the configuration associated with the product or device including one or more competitor products, one or more analysis points, and other information. While the database server 224 is shown as operating on a single server 200 with the marketing message simulation system, it is understood that the database may be located separately on another server or system in network communication with the server 200. The database server 224 may store the historical content data from one or more content systems in an indexed format. There may be a large number of content profiles stored in the database 224, including more than 1,000 or more than 10,000. There may also be a large number of historical content data stored in database 224, including 100s or 1000s of historical datapoints for each content profile.

The historical content in database 224 may be indexed using a collection timestamp by the web scraper 226. The web scraper 226 may, on user request or as a function of a periodic polling of the content systems 112 may make a request to a content system 112 to capture the content (including both text content and other media content) from the content system 112 and store it in the database 224.

The historical content in database 224 may include content entries associated with different markets associated with the pharmaceutical product or medical device. For example, the national content sites of a pharmaceutical company may be individually collected by web scraper 226. The national content sites may include a localized website for the United States, Canada, Germany, England, France, Brazil, etc. for a pharmaceutical product associated with a content profile.

The content entries in database 224 may include text data, media data such as image data, and screenshot data associated with a browser rendering of the website at content system 112. The content entries may include language translations of text data performed by the LLM system 108.

The content entries in database 224 may include content created by the LLM Unit 228 when an LLM response is received from an LLM system such as LLM system 108. For example, content prompts may be executed against a particular content entry by the LLM system 108 and the results may be stored in association with the content entry.

The database 224 may be a file system such as Amazon® Simple Storage Service (S3). The content entries for each particular pharmaceutical product or medical device may be stored in an S3 bucket.

The database 224 may contain a comprehensive document corpus associated with a particular pharmaceutical product or medical device related to pharmaceutical regulatory disclosures. This may include but is not limited to prescribing information, previous medical legal regulatory (MLR) body decisions, and authoritative databases on drug information approved by regulatory bodies like the FDA.

The web scraper 226 may be a software system for connecting to content devices such as content devices 112 (see FIG. 1). The web scraper 226 may be triggered on a regular schedule, or on request of a user using a user interface described herein. The web scraper 226 may further be triggered automatically by a user submitting updated content or “copy” to a content-management system, and then requesting a “scorecard” or “checklist” comparison. The web scraper 226 may be a headless browser based scraper, such as a scraper based on Chromium.

The web scraper 226 may be used to transmit a content collection request to the content devices 112, and storing the results in the database 224 in association with a content profile. The content collection request may be configured based on a content profile stored in the database 224, including for example, one or more URLs associated with the content profile. The one or more URLs may include a first party URL that links to a client website, and one or more third-party URLs that are associated with competitors or other products with similar therapeutic application. The web scraper 226 may further compare the content collection response received based on the content collection request and may store the difference or delta between a prior acquired content entry and a current content entry.

The web scraper 226 may capture static content, image or other media content, javascript content, cascading style sheet content, or other web assets. The web scraper 226 may further store a screenshot of one or more websites associated with the content profile in the database 226.

The web scraper 226 may perform post-scraping processing. Upon scraping, processing may include categorizing the content data (text, images, videos), filtering irrelevant content based on predefined criteria in the content profile configuration, etc. This may improve the relevance of the stored content entries. The web scraper 226 may similarly break the content down into chunks and create vector embeddings associated with the content.

The collected screenshots of the from the content devices 112 may similarly be processed to remove images including the display of page banners.

Images and other media collected by the web scraper 226 are stored in the database 224 and include metadata extraction, enabling the media to be searchable and available for analysis within the data lake. For example, image alt text and surrounding textual content are analyzed to understand the context and relevance may be stored in association with the media. Images may be processed using an OCR program to store the text used within them. PDFs filed may be scraped for their content and may have embedding data created in association with their content.

The web scraper 226 may automatically generate a sitemap associated with the content device it is collecting content from. The sitemap may provide a set of changes across a website that may have changed since the web scraper 226 last collected content. The sitemap may be produced based on pages scraped. The sitemap may include a visual layout, as well as a “delta”, “difference”, or change data including new additions, deletions or edits. This may include calling out any new pages, missing pages, or pages that are remaining but have changed.

The web scraper 226 may further process the scraped content from the content device using the LLM unit 228. For example, the scraped content may be processed using a prompt such as “Identify any non html resources such as pdf files, videos, discussion guides etc. Provide the response in the form of a json {” value “: 5, “detail”: text}. “. These additional non-html resources may be scraped by web scraper 226 and included in the content profile stored in database 224. In an alternate embodiment, the web scraper 224 may provide the response in XML or another markup.

The content profile stored in database 224 may include one or more therapeutic categories of the pharmaceutical product or medical device. The therapeutic category may be one of the US FDA USP Therapeutic Categories.

The one or more 3^{rd} party content devices in the content profile may be identified manually (i.e. by a user providing a URL to a competitor content device), or may be identified automatically based on a therapeutic indication of the content profile. For example, a content profile may indicate that a product is for cholesterol control therapy, and the user interface creation system may identify one or more 3^{rd} party competitor content devices that may be used for comparative analysis.

Similarly, the web scraper 226 may use the therapeutic category provided in the configuration of the content profile to selectively capture content from the content devices. For example, where a single pharmaceutical product (either 1^{st} party of 3^{rd} party) has multiple therapeutic indications for the same product. In practice, this may include “Rheumatoid Arthritis”, and “Crohn's Disease” for a pharmaceutical brand like Humira®. The therapeutic category information may be used in this example to compare content related to Humira® for Crohn's disease against another brand for Crohn's disease, or alternatively compare against all existing information for Humira®.

The LLM Unit 228 provides integration with the LLM system. This may include a variety of LLMs such as those described for LLM system 108 (see FIG. 1).

The LLM Unit 228 may receive a content collection response from the web scraper 226 or the database 224. The LLM unit 228 may receive a content profile including a plurality of content entries from the database 224. The LLM Unit 228 may access a configuration of the content profile which may provide one or more analysis prompts. The LLM unit 228 may execute each of the one or more analysis prompts including the current content entry of the content profile, the historical content entries of the content profile, and prompt language from the configuration in order to perform analysis of the current content entry in view of the historical content.

The function of the analysis prompts may be to evaluate the potential performance of updated content or “copy” that is proposed to be changed on the content device. For example, an editor may change the marketing copy associated with a particular therapeutic effect associated with a pharmaceutical product and may want to analyze the proposed changes to see how the performance may change. This performance may be provided to the user in the form of a user interface such as a checklist or a scorecard.

In another embodiment, the analysis prompts may be executed by the LLM Unit 228 against the LLM system and may provide for summarizing changes over time. The analysis prompt in this case may include the contents of the change, or alternatively content from a first collection event and content from a section collection event. The content from each collection event may be included in the analysis prompt, and a summarization may be provided in the response from the LLM system. For example the web scraper 226 may collect content from a content device at four timestamps, i.e. timestamps 1, 2, 3, and 4. The LLM Unit 228 may send an analysis prompt to the LLM system to summarize the differences over time for different content devices or across different versions of the same content collected at different timestamps from the same content device. For example, to summarize messages for a pharmaceutical product that were in market in Q1 of 2024 for all content devices or for a single content device summarize the differences and trend changes from 2022 to 2024.

The user interface may provide the responses from the LLM unit 228 in a summarized form. The responses may include metrics based on LLM analysis of the updated content itself, or, may provide metrics based on LLM analysis of the updated content compared with historical content of the same product or device, or, may provide metrics based on LLM analysis of the updated content compared with other market participants based on competitors identified and stored in the content profile or based on therapeutic indications identified and stored in the content profile.

The user interface may provide a summary of changes over time. For example, as noted above, when the LLM system summarizes the changes from two different collection events, the user interface may include the content collected in the first event and the content provided in the second event and provide highlighting of the changes and a summary of the content associated with the changes.

The content profile may store an association with a plurality of countries or a plurality of 1^{st} party sites. For example, a particular product may have different regulatory requirements and thus different 1^{st} party content websites made available for every country.

The performance assessment using the user interfaces described herein may assess the updated brand copy or content compared with the other copy or content for other participants in the market for each 1^{st} party national website (and its competitors).

The LLM Unit 228 may connect to different LLM systems 108 (as listed above at reference 108). The LLM unit 228 may dynamically switch LLM systems 108 based on the cost per token and the relative length of a prompt to be sent to the LLM systems 108. The LLM Unit 228 may similarly dynamically switch LLM system 108 based on the prompt used for the analysis, and the context window length as determined based upon the input content.

The LLM Unit 228 may parse the current content entry, as well as the 1^{st} party historical content entries, as well as the 3^{rd} party content entries to identify changes in the content over time. The changes may be included in the prompt to the LLM system 108 in a prompt that can identify changes in the performance of the marketing content over time.

The LLM Unit 228 analysis may include determining a reading level of a content entry. For example, this reading level may be determined based on a Flesch-Kincaid readability tests or other tests providing quantitative metrics of reading level.

These assessments from the LLM unit 228 may be stored in the database 224 in association with the content entry.

The LLM Unit 228 may receive configuration for a content profile that may provide particular regulatory assessments based on regulatory schemes associated with a pharmaceutical program or medical device.

The LLM Unit 228 may receive a content profile configuration to generate a comparative analysis of a 1^{st} party content entry and other 3^{rd} party content entries according to a particular analysis. For example, the configuration may provide an assessment of client A for brand B, based on particular categories of questions. This comparative analysis may include a comparison of the content entry for client A for brand B against third-party B for brand C and third party D for brand E. The present systems and methods may operate to provide an improved scorecard or checklist user interface that provides this analysis to the user.

The LLM Unit 228 may further be used to highlight sections of the screenshots or images that are submitted for analysis. For example, the historical content entries or the current content entry may include a screenshot. The LLM system 108 may be instructed via prompting to highlight particular portions of the screenshot in its response in order to provide an image-based analysis in addition to the other types of analysis described herein. For example, during the text analysis of content if an image banner on the main page of the site within the content has changed, this change may be flagged for visual analysis. The visual analysis may include the image banner changes. The changes may be analyzed by a multi-modal model associated with the LLM Unit 228, the multi-modal model may provide for analysis of image content changes. In one example, if the image banner changes from an individual person to a family including more than one individual person the content of the change may be described by the multi-modal LLM and changed. The flagged image content change may also be including identifying where parts of an image are visually highlighted to increase the prominence or placement of a block of information moved within the image conent.

For example, for a pharmaceutical product an analysis prompt provided in the content profile configuration may include a prompt to analysis the historical trend in the treatment of sodium, and associated side effects that the product may have with respect to the patient's sodium intake. For example, this pharmaceutical product may be configured using analysis prompts such as “How is the efficacy communicated?” which may be used to provide a comparative analysis using the LLM Unit 228. The analysis prompts may cover other questions also, such as “Is Financial Assistance or Support presented?”. When the LLM system responds to content these prompts may be run each time, or each time new content changes are identified.

In at least one embodiment, there may be brand specific questions and brand-specific considerations about the pharmaceutical product or its identified competitors. For example, for a pharmaceutical product that is high in sodium content, a patient using such a medication can face risks or side effects. In this case, comparing content information details on this brand-specific concern is communicated in the content associated with the pharmaceutical product and its competitors are important considerations for marketing content associated with the pharmaceutical product. Then, for this pharmaceutical product or collection of brands, and identified competitors, the LLM Unit 228 may use analysis prompts to provide a specific LLM based assessment in addition to the standard analysis prompts.

The LLM system may respond to the LLM unit 228 as specified in the content profile configuration. This may include the format of the data, and the type of resultant response. The format of the response from the LLM system 108 to the LLM unit 228 may be in json, XML, CSV, or another format. The type of resultant response may include numerical (e.g. integer-based, float-based, double precision, etc.), categorical (e.g. type A, B or C), a range of values (e.g. 1-2, 3-4, 10-20, etc.), a scientific notation (e.g. 5×105 etc.), a text-based value (e.g. “George, Ringo, John, Paul”), boolean (e.g. true or false, 1 or 0, etc) or other types of values.

For example, the prompt executed in order to provide an evaluation of the handling of sodium intake may specify that the LLM system 108 return the response in a particular format such as json or XML, and may provide the resultant analysis as a binary value.

The analysis of the content using LLM Unit 228 may occur manually upon request of a user, or alternatively, may occur automatically when the web-scraper 226 performs its scheduled collection. The collection of content entries 226 may automatically trigger the LLM Unit 228 to perform the analysis in the content profile configuration and may automatically update the respective user interfaces and automatically store the analysis as well as the new content items in the database 224.

The user interfaces created by the user interface engine 230 may also include a text box that allows a user to ask an ad-hoc question based on a prior evaluation. For example, the user may have a particular question about an evaluation provided in the user interface and may want further information. The particular ad-hoc questions asked by users may be stored in the database 224 and may be used to automatically create new analysis questions in a content profile configuration where similar questions are frequently.

The user interface engine 230 is configured to generate interfaces for users to configure marketing message simulation, request a marketing message simulation, view marketing message simulation results, send and receive marketing message simulation results, etc. The various interfaces generated by the user interface engine 230 may be transmitted to a user device by virtue of the Web/API Unit 232 and the network unit 204.

The processor unit 208 can also execute a user interface (UI) creation engine 230 that is used to generate various UIs for delivery via a web application provided by the Web/API Unit 232, some examples of which are shown and described herein, such as interfaces shown in FIGS. 3A-3E, 4A-4F and 5A-5E.

The Web/API Unit 232 may be a web-based application or Application Programming Interface (API) such as a REST (REpresentational State Transfer) API. The API may communicate in a format such as XML, JSON, or other interchange format.

The Web/API Unit 232 may receive a user request for content evaluation, or may similarly receive a request from a user for a user interface as described herein. The Web/API Unit 232 may apply methods herein to determine an evaluation based on the user request, and then may provide the results in a user interface as provided herein. The evaluation may be stored after receipt of a user request in database server 224. The Web/API Unit 232 may provide the user interface (such as those in FIGS. 3A-3E, 4A-4F and 5A-5E) to the user via network unit 204.

Referring next to FIGS. 3A-3E together, there is shown a user interface diagram 300 in accordance with one or more embodiments. The user interface generation system may generate a scorecard user interface 300 for comparing different brands based on predefined analysis criteria (which may be stored in the content profile configuration). This may include brand messaging, audience targeting, financial support programs, focus on mechanism of action or delivery, and accessibility based on content reading level. The user interface 300 may be provided in a browser window 302 or using a software application saved on a user device that may interact with an API of the user interface generation system.

As noted above, a web scraper may access a content device to load content for analysis (e.g. web scraper 226). This content may be stored in database 224 in association with the content profile.

The content profile contains configuration that includes one or more analysis prompts that may be run against the received updated content, the historical 1^{st} party content, and the 3^{rd} party content. The analysis prompts may be one or more LLM prompts that LLM unit 228 may run using an LLM system 108 (see FIG. 1). The responses to the analysis prompts may be stored together in the database 224. The analysis prompts may request responses from the LLM system 108 (e.g. FIG. 1) are formatted in javascript object notation (json), for example, by prompting “Please just give me the json, don't include any preamble, don't wrap it in a “′json block”. In an alternate embodiment, the response may be provided in XML or another markup.

The user interface 300 may be generated by the user interface engine 230 (e.g. FIG. 2) once the LLM unit 228 receives a response from the LLM system 108.

The scorecards may be generated by applying machine learning models to the scraped content data from the LLM system 108 (see FIG. 1) by the LLM unit 228, scoring brands on various criteria. The scorecard may use natural language generation (NLG) to summarize findings and present them in an accessible format.

The responses to the prompts submitted queries may be used to generate the scorecard 300. The combined json (or XML, or another markup) from the responses to the prompts may be combined together and provided to the user interface for rendering the data.

The LLM-assisted scorecard is presented to the user through a user-friendly interface 300, enabling them to easily compare multiple brands and identify areas for improvement.

As shown, a 1^{st} party pharmaceutical product is compared in the first column 320 to a 3^{rd} party pharmaceutical product in the second column 340. The comparison may include a reading level 322 of the 1^{st} party product content compared with a reading level 342 of the 3^{rd} party product content.

The reading level 322 for the 1^{st} party product content 351 and reading level 342 for the 3^{rd} party product content 352 may be determined based on the combined scraped content data using a natural language processing algorithm. The determined reading level 322 for the 1^{st} party product content 351 and reading level 342 for the 3^{rd} party product content 352 may be stored in database 224 in association with the content profile. The reading level may include a determined reading ease, a grade level, a total number of words, a sentence count, an average sentence length, a syllable count, and an average syllables per word. The reading level 322 and 342 may further include an overall reading level score.

The comparison may include financial assistance content analysis 324 of the 1^{st} party product content 351 compared with financial assistance content analysis 344 of the 3^{rd} party product content 352.

The financial assistance content analysis 324 of the 1^{st} party product 351 and the financial assistance content analysis 342 for the 3^{rd} party product content 352 may be generated by the LLM system 108 (see FIG. 1) via the LLM unit 228 using a prompt for each containing the scraped content data and a prompt such as “Does the site say anything about cost, access, savings, copay or co-pay programs, financial assistance or financial support, provide the details?. Provide the response in the form of a json {” value “: True/False, “detail”: text} “. The response based on this prompt may be stored in the database 224 in association with the content profile. The financial assistance content analysis 324 and 344 may include a text-based analysis of the site content of the respective content device. In an alternate embodiment, the response may be provided in XML or another markup.

The comparison may include mechanism of action content analysis 326 of the 1^{st} party product content 353 compared with mechanism of action content analysis 346 of the 3^{rd} party product content 354.

The mechanism of action content analysis 326 of the 1^{st} party product content 353 and the mechanism of action content analysis 346 of the 3^{rd} party product content 354 may be generated by the LLM system 108 (see FIG. 1) via the LLM unit 228 using a prompt for each containing the scraped content data and a prompt such as “Is there a mechanism of action mentioned, provide a summary of the details of the mechanism of action. Provide the response in the form of a json {” value “: True/False, “detail”: text} “. The response based on this prompt may be stored in database 224 in association with the content profile. The mechanism of action content analysis 326 and 346 may include a text-based analysis of the site content of the respective content device. In an alternate embodiment, the response may be provided in XML or another markup.

The comparison may include mechanism of delivery content analysis 328 of the 1^{st} party product content 353 compared with mechanism of delivery content analysis 348 of the 3^{rd} party product content 354.

The mechanism of delivery content analysis 328 of the 1^{st} party product content 353 and the mechanism of delivery content analysis 348 of the 3^{rd} party product content 354 may be generated by the LLM system 108 (see FIG. 1) via the LLM unit 228 using a prompt for each containing the scraped content data and a prompt such as “Does the site mention how the drug is delivered, whether through intravenous or oral delivery? If the delivery type is injection or intravenous then return “far: syringe”, if it\'s oral then return “far: tint”, if it\'s a pill or tablet then return “far: pills”. Summarize the details of the mechanism of delivery. Provide the response in the form of a json {“value”: delivery_type, “detail”: text} “.

The response based on this prompt may be stored in database 224 in association with the content profile. The mechanism of delivery content analysis 328 and 348 may include a text-based analysis of the site content of the respective content device. In an alternate embodiment, the response may be provided in XML or another markup.

The comparison may include a call to action analysis (not shown) of the 1^{st} party product content compared with a call to action analysis (not shown) of the 3^{rd} party product content.

The call to action analysis of the 1^{st} party product content and the call to action analysis of the 3^{rd} party product content may be generated by the LLM system 108 (see FIG. 1) via the LLM unit 228 using a prompt for each containing the scraped content data and a prompt such as “Ignoring details in the shared footer of every page, give the number of call to actions and a summary of the calls to actions for the pages in english. Provide the response in the form of a json {” value “: 5, “detail”: text}. “. The response based on this prompt may be stored in database 224 in association with the content profile. The call to action analysis may include a text-based analysis of the site content of the respective content device. In an alternate embodiment, the response may be provided in XML or another markup.

The user interface 300 may similarly include a user interface element such as a select box (not shown) that includes a historical listing of content captures (i.e. scrapes) of the content device that are stored in database 224. There may be one user interface element for each column permitting a user to select a particular set of content collected at a particular point in time for comparison.

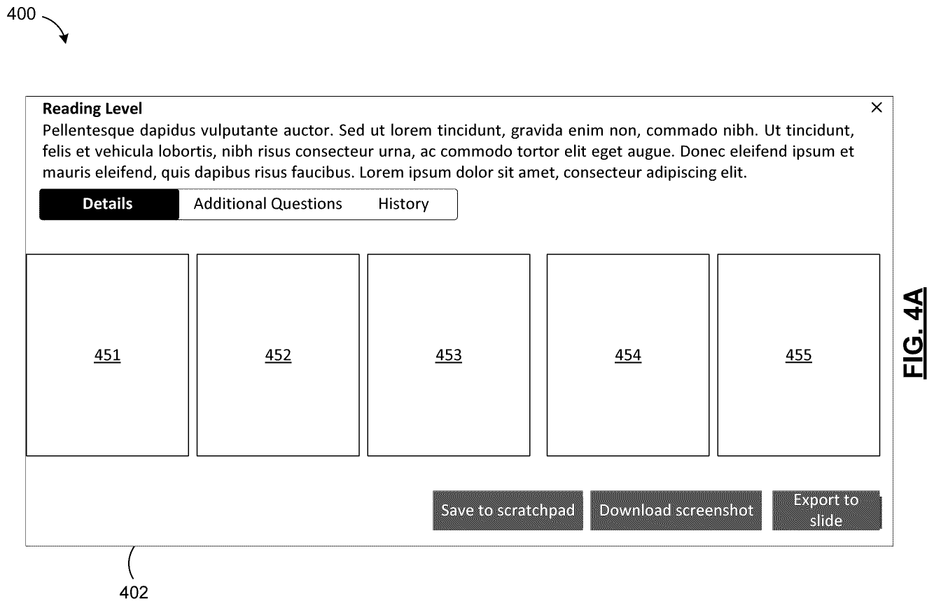

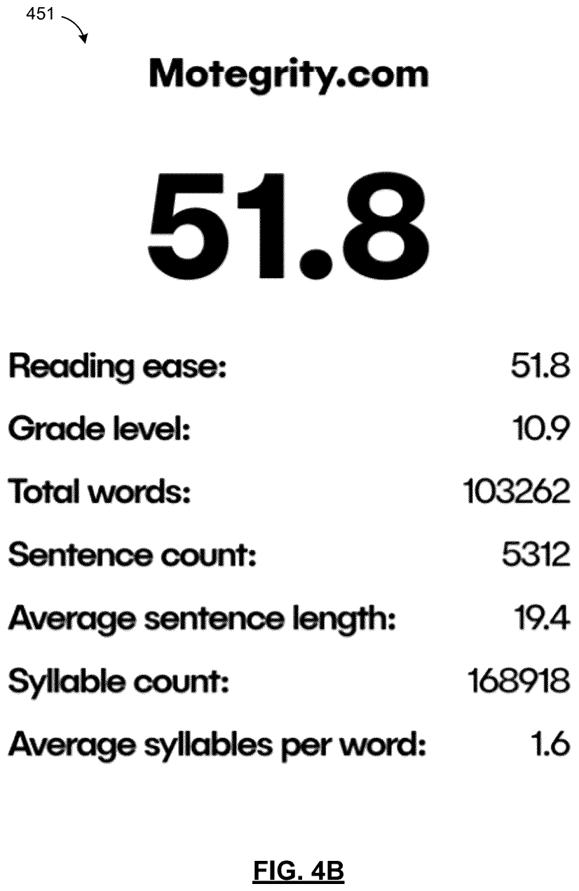

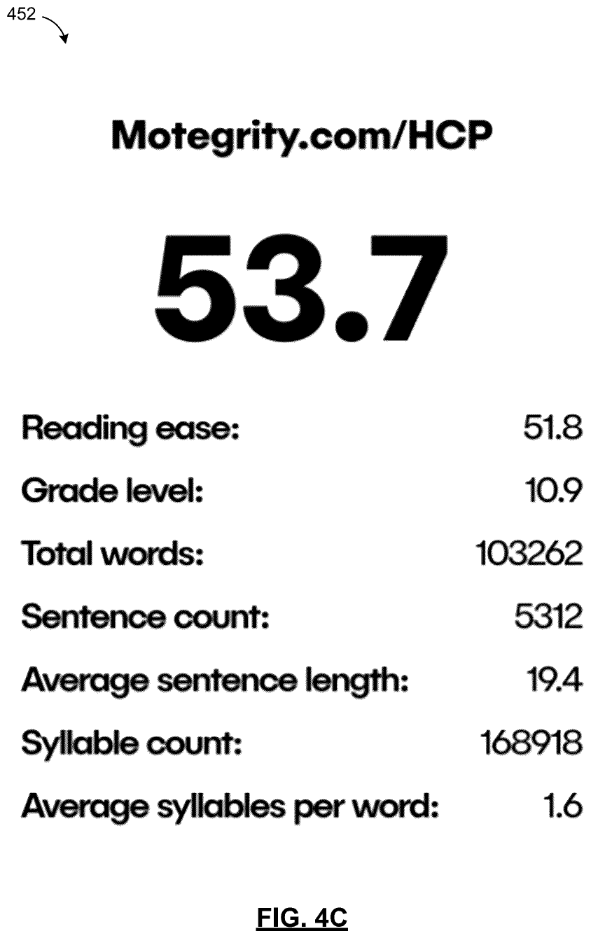

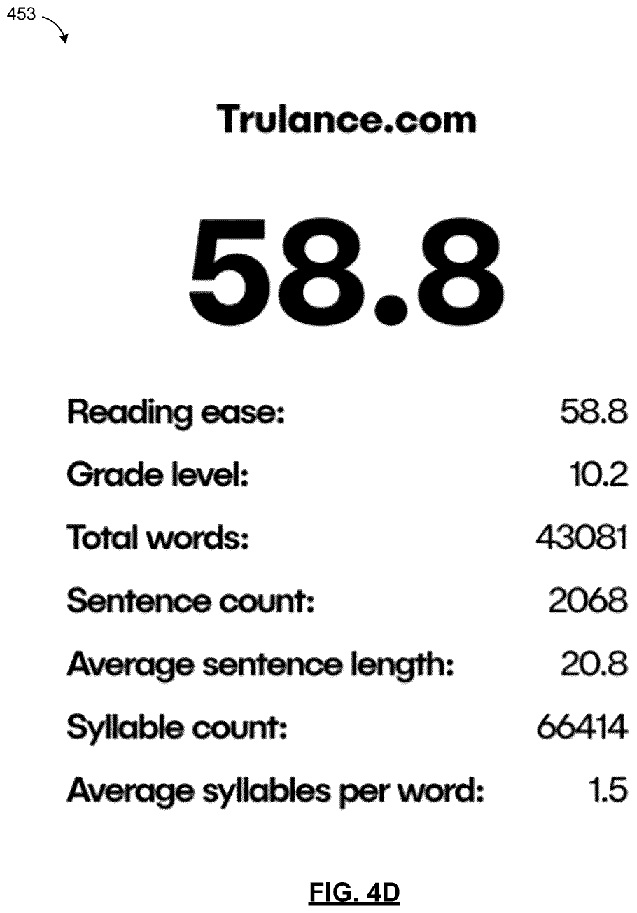

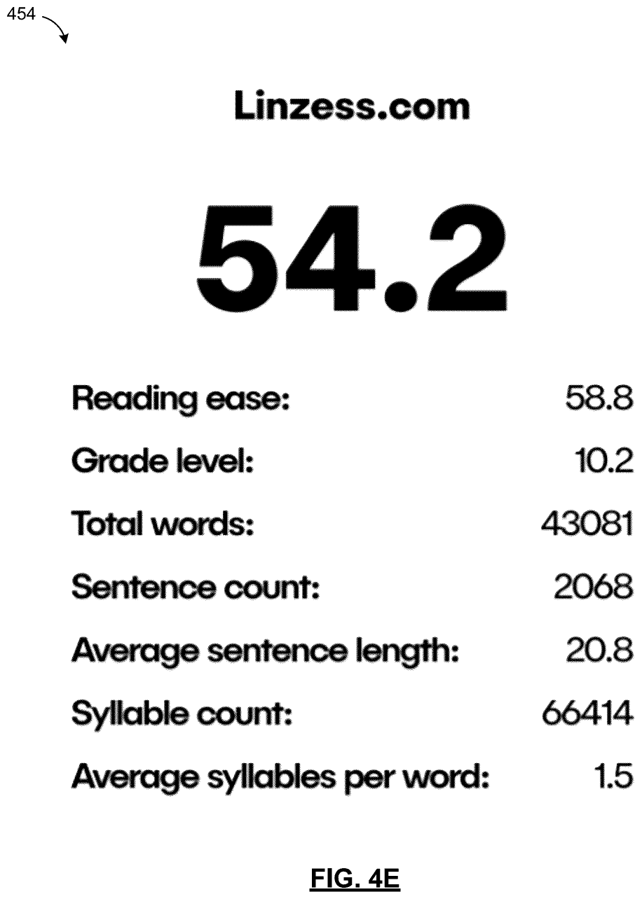

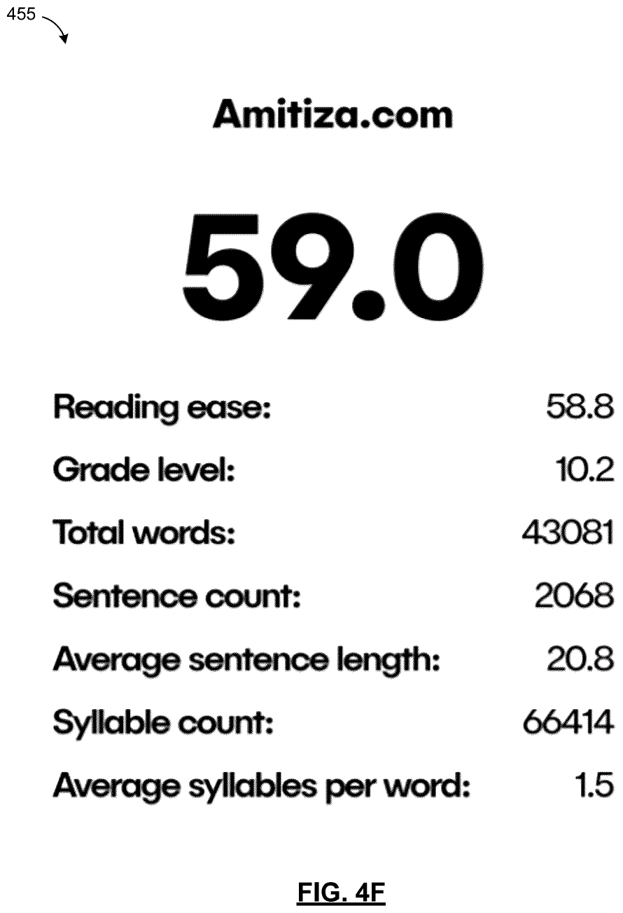

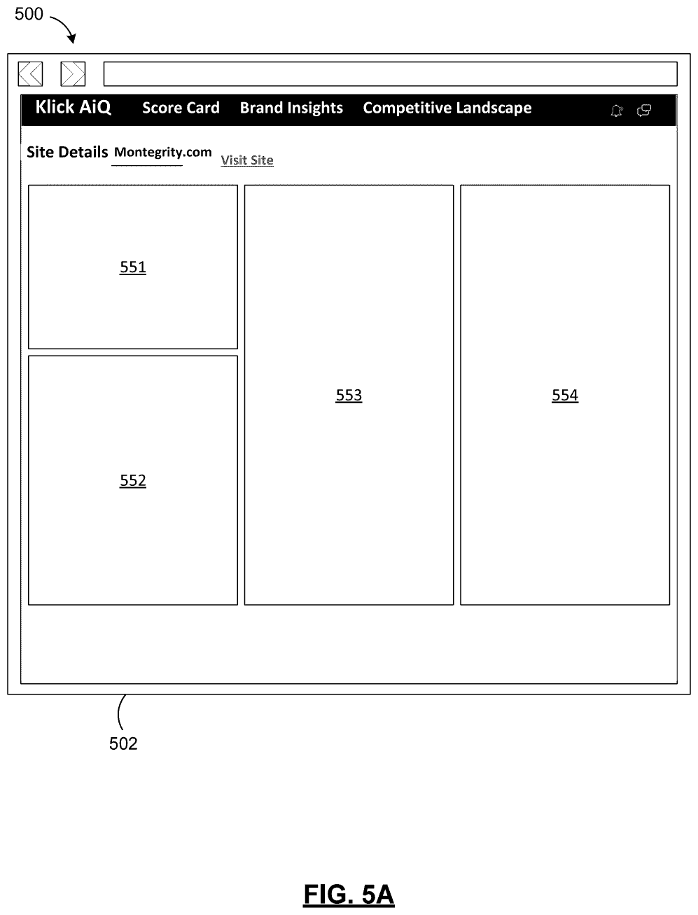

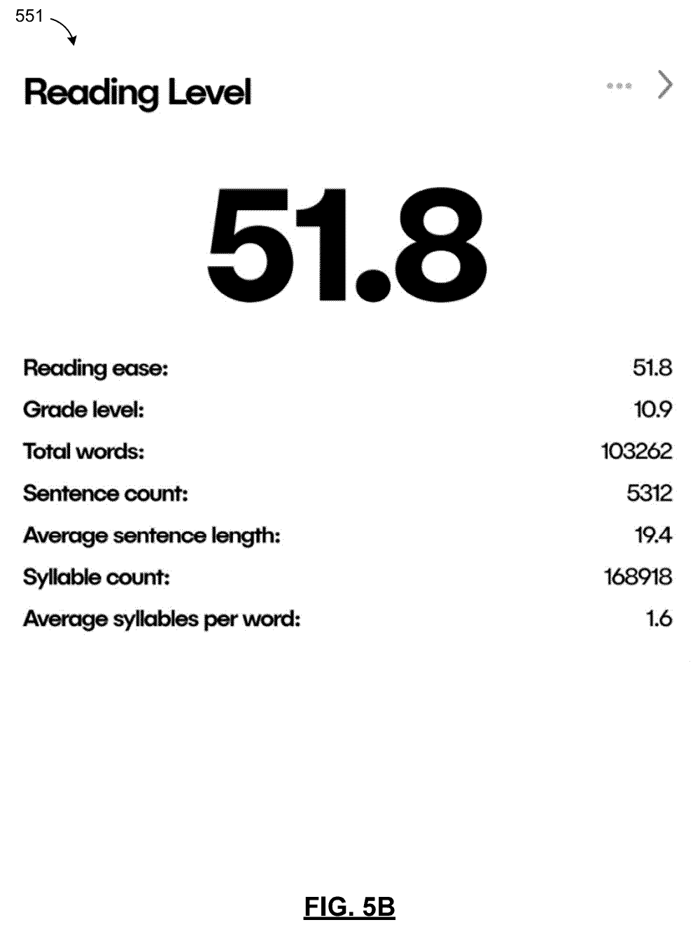

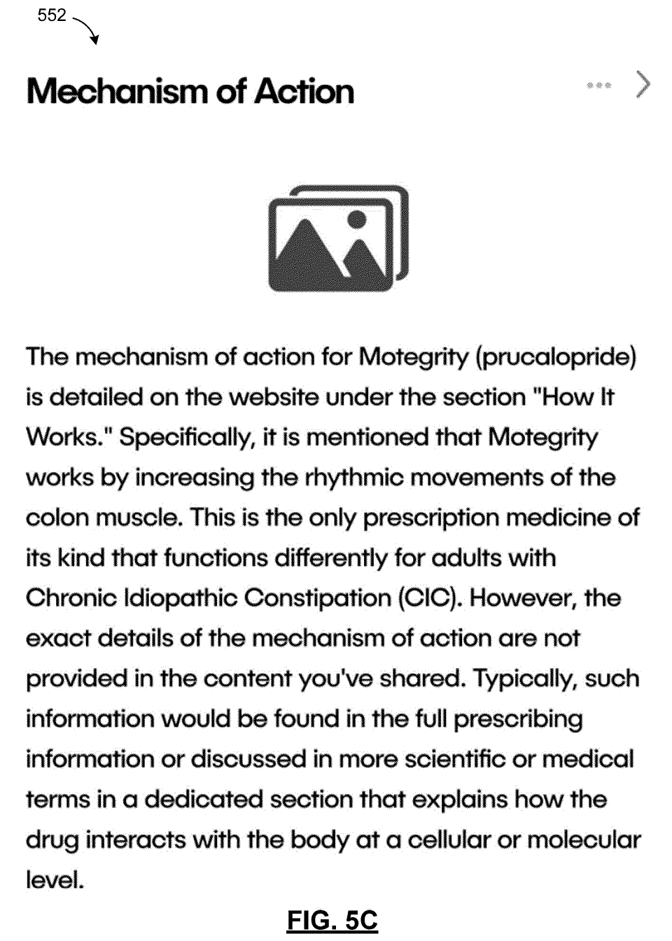

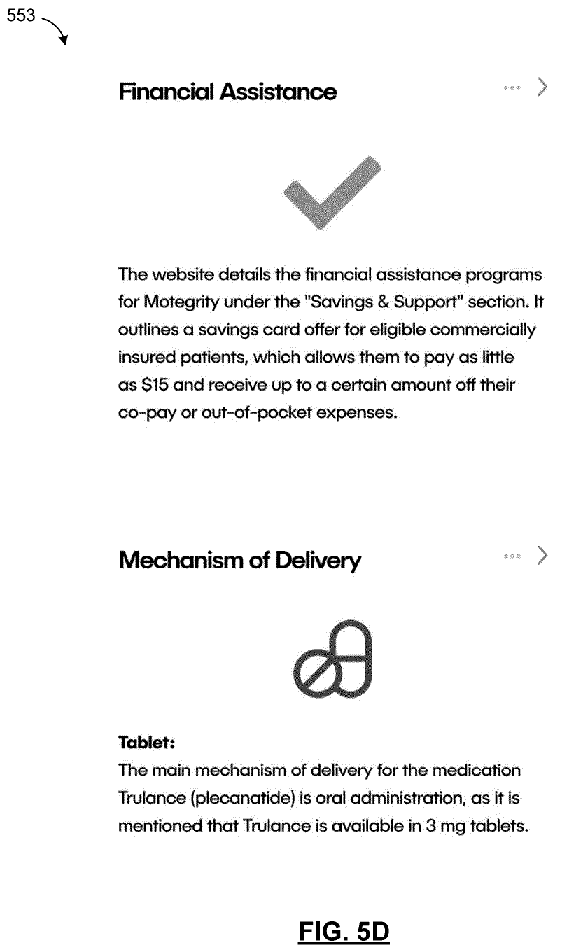

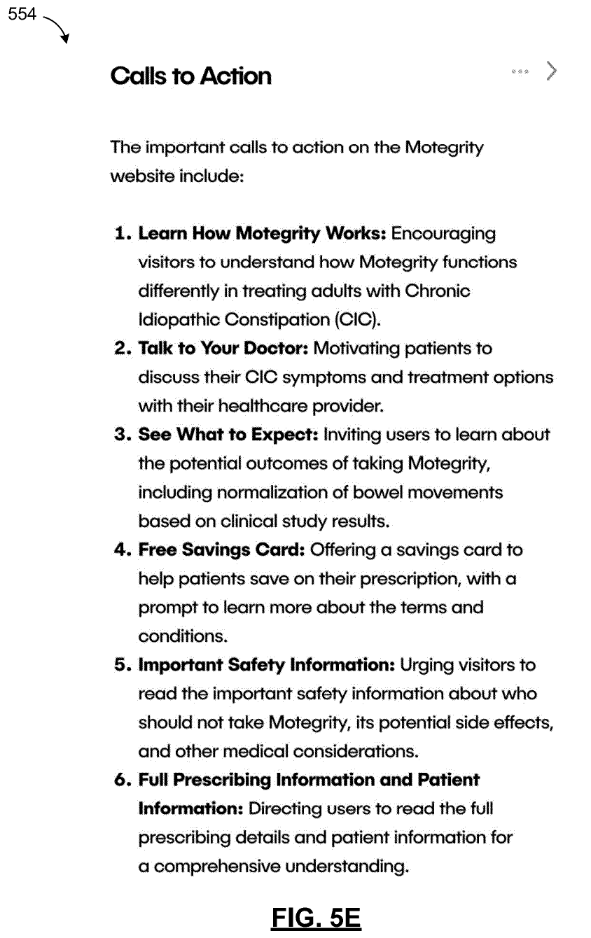

The content analysis portions of the user interface 300 may be user selectable, and may show a secondary user interface such as a broader comparison of 3^{rd} party reading level as shown in FIGS. 4A-4F, or a full report for the product such as the full report interface shown in FIGS. 5A-5E.

The scorecard interface 300 may further provide a text box or text input control 360 for a “conversational comparison” including a submission button 362. The conversational comparison enables somebody to ask an arbitrary question about the 1^{st} party and 3^{rd} party sites on the respective content devices and compare them. The conversation comparison may be built by the LLM Unit 228 making multiple requests to an LLM system 108 for an analysis of each site content, and then a subsequent request to an LLM system may be made to compare the analysis generated for each site content. The conversational comparison request from a user may be responded to using the response to the subsequent request to the LLM system.

The user may also select from each analysis portion a link that may provide a highlighted screenshot showing elements of the screenshot that provide the basis for the analysis, or further explain why a particular analysis is provided. These highlighted screenshots may be used by a user in a slide deck or presentation for example.

For example, the screenshot highlighting may, for the mechanism of action analysis, identify where on a content page text associated with the analysis is shown by highlighting it in a color or in a box. Images and video on the content scraped from the content device (including from a 1^{st} party product content or the 3^{rd} party product content or both) can be highlighted as well.

Referring to FIGS. 4A-4F together shows a reading level comparison user interface diagram 400 in accordance with one or more embodiments. The user selection of the reading level content analysis 322 or 342 in FIGS. 3B and 3C may provide the user with a subsequent user interface with a broader comparison of reading level for the content entry. The user interface 400 may be provided in a browser window 402 or using a software application saved on a user device that may interact with an API of the user interface generation system.

The user interface 400 may provide a broader comparison of the different 3^{rd} party content 451-455 associated with other products designed for the same therapeutic indication.

Referring next to FIGS. 5A-5E together, there is shown a detailed user interface diagram 500 in accordance with one or more embodiments. The user interface 500 may be provided in a browser window 502 or using a software application saved on a user device that may interact with an API of the user interface generation system.

The detail view 500 may be provided by a user selecting a view details link in the 1^{st} party product content column 320 (e.g. FIG. 3A). The detail view 500 may provide further detailed analysis of the content entry.

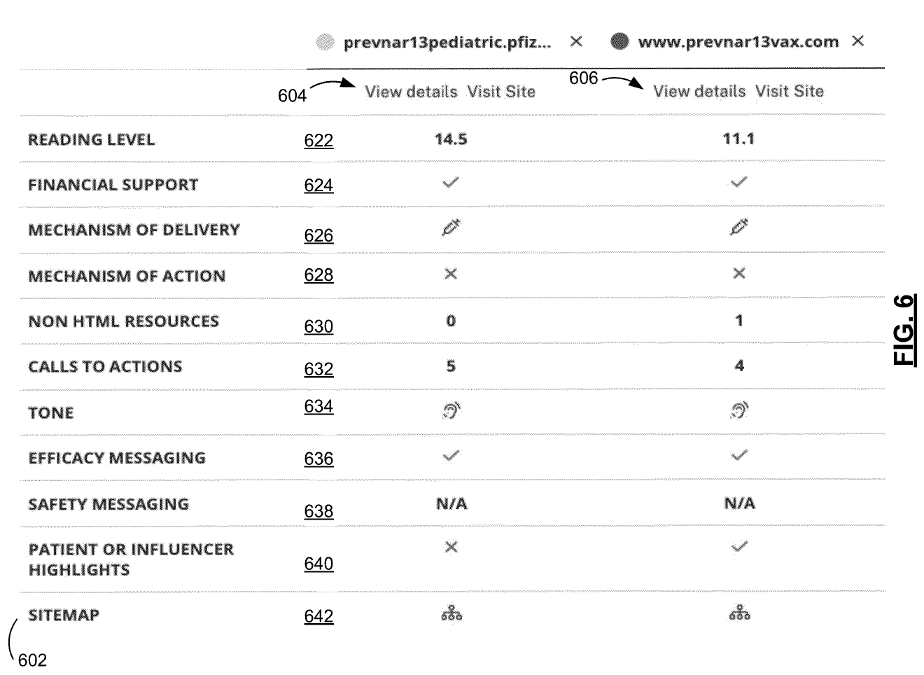

Referring next to FIG. 6, there is shown a checklist user interface diagram 600 in accordance with one or more embodiments. The checklist user interface 600 may include similar content analysis as in FIGS. 3A-3E. For example, the reading level content analysis 622 may correspond to reading level content analysis 322, the financial support content analysis 624 may correspond to the financial support content analysis 324, the mechanism of action content analysis 626 may correspond to the mechanism of action content analysis 326.

The checklist 600 may include the analysis provided in comparison interface 300, including for example reading level analysis 622, financial support analysis 624, mechanism of delivery analysis 626, mechanism of action analysis 628, non-html resources analysis 630, and calls to action analysis 632. As noted, the prompts to the LLM system 108 via LLM unit 228 may include a categorization of the analysis (for example, Boolean, categorical, numerical, etc.). Boolean categorization may provide a boolean value displayed within the user interface as a checkmark or “x”. Categorical categorization may provide an icon corresponding to the identified category of the analysis for the content.

The reading level analysis 622 may be represented by the overall reading level numerical score.

The financial support analysis 624 may be represented by a Boolean, shown as a check or an “x”.

The mechanism of delivery 626 may be represented by an icon, for example, a syringe or a pill categorization.

The mechanism of action 628 may be represented by a Boolean, shown as a check or an “x”.

In addition to the analysis from comparison interface 300 a tone analysis 643, efficacy messaging analysis 636, safety messaging analysis 638, patient or influencer highlight analysis 640 and sitemap analysis 642 of the 1^{st} party product content and the 3^{rd} party product content may be determined.

The tone analysis 634 of the 1^{st} party product content and the 3^{rd} party product content may be represented by an icon. The tone analysis 634 may be selectable by the user in the interface that may display a text-based response to the prompt.

The tone analysis 634 of the 1^{st} party product content and the 3^{rd} party product content may be generated by the LLM system 108 (see FIG. 1) via the LLM unit 228 using a prompt for each containing the scraped content data and a prompt such as “What is the tone of the content? Include at least specific excerpts of text that were used to make this determination and the pages these were found on. Exclude the ISI or Important Safety

Information section from this assessment. Always use “far: ear-listen” for the value. Provide the response in the form of a json {“value”: “far: ear-listen”, “detail”: text} “. The response based on this prompt may be stored in database 224 in association with the content profile. The tone analysis 634 may include a text-based analysis of the site content of the respective content device. In an alternate embodiment, the response may be provided in XML or another markup.

The efficacy analysis 636 of the 1^{st} party product content and the 3^{rd} party product content may be represented by an icon. The efficacy analysis 636 may be selectable by the user in the interface that may display a text-based response to the prompt.

The efficacy analysis 636 of the 1^{st} party product content and the 3^{rd} party product content may be generated by the LLM system 108 (see FIG. 1) via the LLM unit 228 using a prompt for each containing the scraped content data and a prompt such as “Does the site discuss the efficacy of the drug? If it does how prominently and frequently is efficacy discussed. What are the main points made as they relate the drugs efficacy. Provide the response in the form of a json {” value “: True/False, “detail”: text} “. The response based on this prompt may be stored in database 224 in association with the content profile. The efficacy analysis 636 may include a text-based analysis of the site content of the respective content device. In an alternate embodiment, the response may be provided in XML or another markup

The safety analysis 638 of the 1^{st} party product content and the 3^{rd} party product content may be represented by an icon. The safety analysis 638 may be selectable by the user in the interface that may display a text-based response to the prompt.

The safety analysis 638 of the 1^{st} party product content and the 3^{rd} party product content may be generated by the LLM system 108 (see FIG. 1) via the LLM unit 228 using a prompt for each containing the scraped content data and a prompt such as “Does the site discuss the safety of the drug? If it does how prominently and frequently is safety discussed. What are the main points made as they relate the drugs safety. Provide the response in the form of a json {” value “: True/False, “detail”: text} “. The response based on this prompt may be stored in database 224 in association with the content profile. The safety analysis 638 may include a text-based analysis of the site content of the respective content device. In an alternate embodiment, the response may be provided in XML or another markup.

The patient or influencer highlights analysis 640 of the 1^{st} party product content and the 3^{rd} party product content may be represented by an icon. The patient or influencer highlights analysis 640 may be selectable by the user in the interface that may display a text-based response to the prompt.

The patient or influencer highlights analysis 640 of the 1^{st} party product content and the 3^{rd} party product content may be generated by the LLM system 108 (see FIG. 1) via the LLM unit 228 using a prompt for each containing the scraped content data and a prompt such as “Does the site highlight patients through patient testimonials or highlight influencers? If it does provide, what format are these testimonials in and what are the main themes. Provide at least 5 examples. Provide the response in the form of a json {” value “: True/False, “detail”: text} “. The response based on this prompt may be stored in database 224 in association with the content profile. The patient or influencer highlights analysis 640 may include a text-based analysis of the site content of the respective content device. In an alternate embodiment, the response may be provided in XML or another markup.

The sitemap 642 may be user selectable and may provide an interface showing a visual representation of the sitemap from the 1^{st} party product content site and the 3^{rd} party product content site.

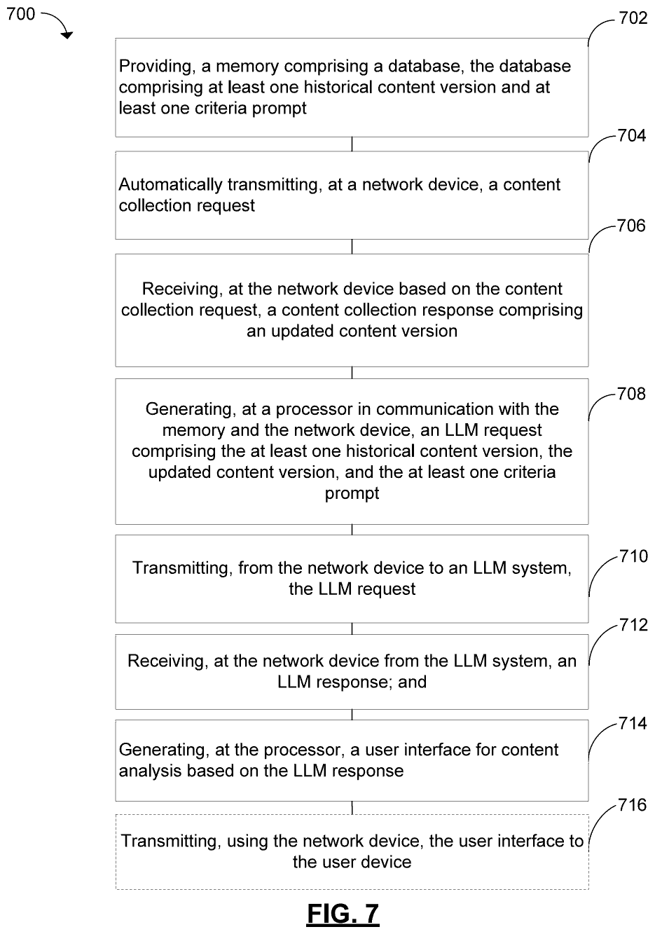

Referring next to FIG. 7, there is shown a method diagram 700 in accordance with one or more embodiments. The computer-implemented method 700 is for generating a large language model (LLM)-based user interface for a user at a user device.

At 702, a memory comprising a database is provided, the database comprising at least one historical content version and at least one criteria prompt.

At 704, a network device automatically transmits a content collection request.

At 706, a content collection response is received at the network device based on the content collection request, the content collection response comprising an updated content version.

At 708, an LLM request comprising the at least one historical content version is generated at a processor in communication with the memory and the network device, the updated content version, and the at least one criteria prompt.

At 710, the LLM request is transmitted from the network device to an LLM system.

At 712, an LLM response is received at the network device from the LLM system.

At 714 a user interface for content analysis based on the LLM response is generated at the processor.

Optionally, at 716, the method may include transmitting, using the network device, the user interface to the user device.

Optionally, the method may further include: transmitting, using the network device, the user interface to the user device.

Optionally, the transmitting of the content collection request and the receiving of the content collection response may be performed using a web scraper.

Optionally, the web scraper may receive text-based data and image-based data in the content collection response.

Optionally, the method may further include: generating, using the web-scraper, a screenshot.

Optionally, the method may further include: storing, in the database, the screenshot, the text-based data, and the image-based data as a new historical content version, the new historical content version indexed with a timestamp.

Optionally, a first content version of the at least one historical content version may be associated with a first timestamp, and the updated content version is associated with a second timestamp.

Optionally, the method may further include: comparing, at the processor, the first content version and the updated content version to determine a content difference; and wherein the generating at the processor, the LLM request may comprise the content difference.

Optionally, the user interface may comprise a scorecard user interface, the scorecard user interface comprising at least one scorecard metric for each of the at least one criteria prompt, the scorecard user interface may be updated automatically based on the received updated content version.

Optionally, the user interface may comprise a checklist user interface, the checklist user interface comprising at least one checklist metric for each of the at least one criteria prompt, the checklist user interface may be updated automatically based on the received updated content version.

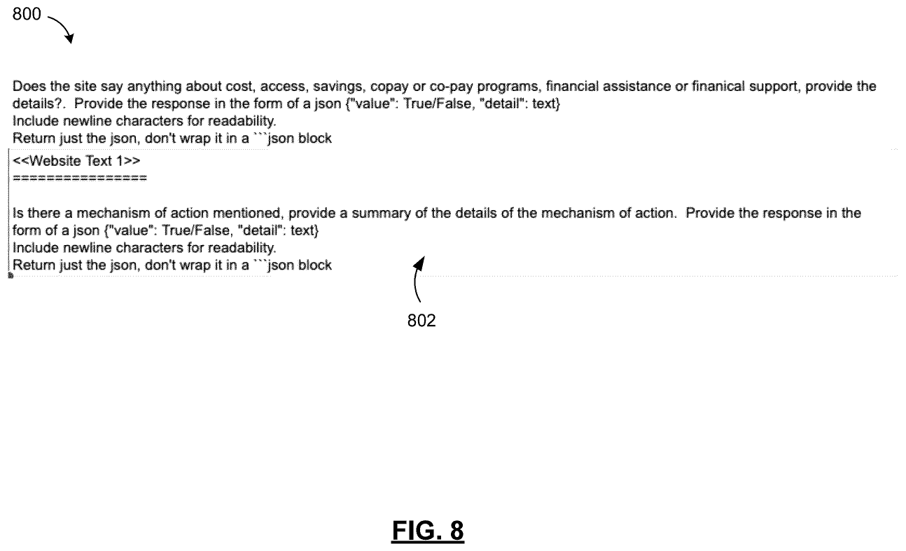

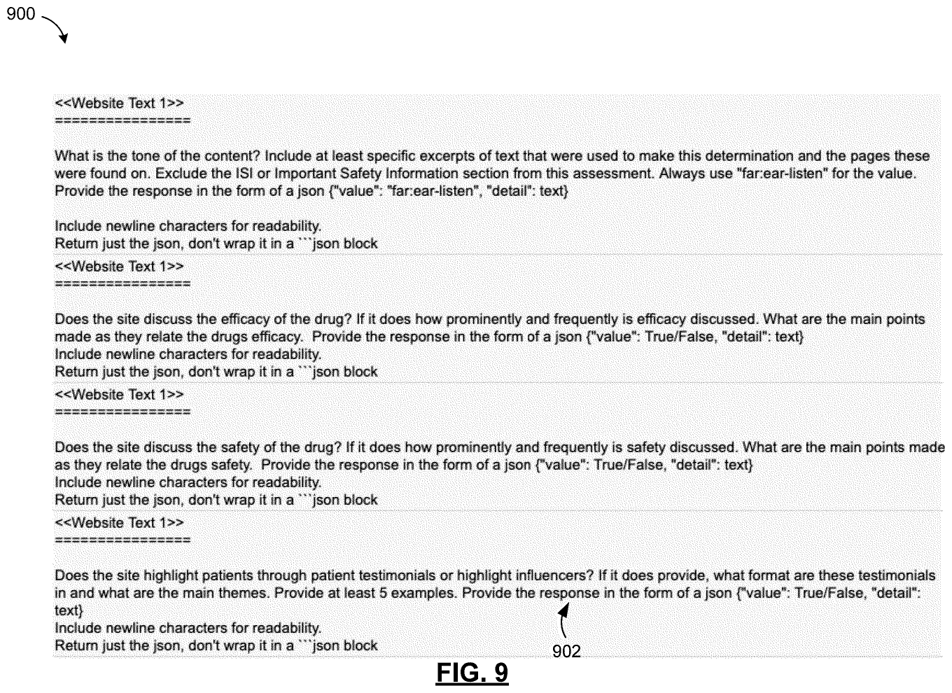

Referring next to FIGS. 8 and 9 together, there is shown two examples of content profile configurations 802 and 902, relating to content analysis prompts that may be used to provide analysis of content entries as discussed herein.

The present invention has been described here by way of example only. Various modification and variations may be made to these exemplary embodiments without departing from the spirit and scope of the invention, which is limited only by the appended claims.

1. A computer-implemented method for generating a large language model (LLM)-based user interface for a user at a user device, comprising:
providing, a memory comprising a database, the database comprising at least one historical content version and at least one criteria prompt;
automatically transmitting, at a network device, a content collection request;
receiving, at the network device based on the content collection request, a content collection response comprising an updated content version;
generating, at a processor in communication with the memory and the network device, an LLM request comprising the at least one historical content version, the updated content version, and the at least one criteria prompt;
transmitting, from the network device to an LLM system, the LLM request;
receiving, at the network device from the LLM system, an LLM response; and
generating, at the processor, a user interface for content analysis based on the LLM response.

2. The method of claim 1, further comprising:
transmitting, using the network device, the user interface to the user device.

3. The method of claim 2, wherein the transmitting of the content collection request and the receiving of the content collection response are performed using a web scraper.

4. The method of claim 3, wherein the web scraper receives text-based data and image-based data in the content collection response.

5. The method of claim 4, further comprising generating, using the web-scraper, a screenshot.

6. The method of claim 5, further comprising storing, in the database, the screenshot, the text-based data, and the image-based data as a new historical content version, the new historical content version indexed with a timestamp.

7. The method of claim 6, wherein a first content version of the at least one historical content version is associated with a first timestamp, and the updated content version is associated with a second timestamp.

8. The method of claim 7, further comprising:
comparing, at the processor, the first content version and the updated content version to determine a content difference; and
wherein the generating at the processor, the LLM request comprises the content difference.

9. The method of claim 8, wherein the user interface comprises a scorecard user interface, the scorecard user interface comprising at least one scorecard metric for each of the at least one criteria prompt, the scorecard user interface updated automatically based on the received updated content version.

10. The method of claim 9, wherein the user interface comprises a checklist user interface, the checklist user interface comprising at least one checklist metric for each of the at least one criteria prompt, the checklist user interface updated automatically based on the received updated content version.

11. A system for generating an large language model (LLM)-based user interface for a user at a user device, comprising:
a memory comprising a database, the database comprising at least one historical content version and at least one criteria prompt;
a network device for:
transmitting a content collection request;
receiving a content collection response based on the content collection request, the content collection response comprising an updated content version;
transmitting to an LLM system, an LLM request; and
receiving from the LLM system, an LLM response, and
a processor in communication with the memory and the network device, the processor configured to:
generate the LLM request comprising the at least one historical content version, the updated content version, and the at least one criteria prompt;
generate a user interface for content analysis based on the LLM response.

12. The system of claim 11, wherein the processor is further configured to:
transmit, using the network device, the user interface to the user device.

13. The system of claim 12, wherein the transmitting of the content collection request and the receiving of the content collection response are performed using a web scraper.

14. The system of claim 13, wherein the web scraper receives text-based data and image-based data in the content collection response.

15. The system of claim 14, wherein the processor is further configured to generate, using the web-scraper, a screenshot.

16. The system of claim 15, wherein the processor is further configured to store, in the database, the screenshot, the text-based data, and the image-based data as a new historical content version, the new historical content version indexed with a timestamp.

17. The system of claim 16, wherein a first content version of the at least one historical content version is associated with a first timestamp, and the updated content version is associated with a second timestamp.

18. The system of claim 17, wherein the processor is further configured to:
compare the first content version and the updated content version to determine a content difference; and
wherein the processor is further configured to generate the LLM request comprises the content difference.

19. The system of claim 18, wherein the user interface comprises a scorecard user interface, the scorecard user interface comprising at least one scorecard metric for each of the at least one criteria prompt, the scorecard user interface updated automatically based on the received updated content version.

20. The system of claim 19, wherein the user interface comprises a checklist user interface, the checklist user interface comprising at least one checklist metric for each of the at least one criteria prompt, the checklist user interface updated automatically based on the received updated content version.
